(19) United States
(12) Patent Application Publication
(10) Pub. No.: US 2026/0137883 A1
(43) Pub. Date: May 21, 2026

(54) WEARABLE RESPIRATOR FIT ASSESSMENT SYSTEM

(71) Applicant: Consequent Labs LLC, Tucson, AZ (US)

(72) Inventor: James Knitter, Oro Valley, AZ (US)

(21) Appl. No.: 19/397,414

(22) Filed: Nov. 21, 2025

Related U.S. Application Data
(60) Provisional application No. 63/723,132, filed on Nov. 21, 2024.

(51) Int. Cl. A61M 16/00; A61M 16/06

(52) U.S. Cl. CPC A61M 16/0003; A61M 16/0683

(57) ABSTRACT

A wearable respirator fit assessment system for real-time fit testing and continuous monitoring of respirators. The system includes a wearable device that attaches to the respirator and incorporates multimodal sensors, including pressure and temperature sensors, for detecting variations in the respirator's dead space. An embedded microcontroller processes the sensor data using a machine learning model running on the device to classify respirator fit without needing any other hardware. The system provides immediate feedback to users through visual, auditory, and haptic notifications and can communicate fit data to a connected user device via Bluetooth or other wireless protocols. A companion mobile application guides users through fit testing exercises, displays real-time results, and provides educational resources for proper respirator use and maintenance. The system also supports continuous fit monitoring during respirator use, enabling users to receive alerts for seal degradation or improper fit.

### CROSS-REFERENCE TO RELATED APPLICATIONS

This application in a non-provisional application and claims priority to U.S. provisional application no. 63/723,132, filed on Nov. 21, 2024, titled Wearable Respirator Fit Assessment System, which is incorporated by reference in its entirety.

### BACKGROUND

The efficacy of respiratory protective equipment, such as filtering facepiece respirators (FFRs), elastomeric respirators, and gas masks, relies heavily on proper fit to ensure a secure seal between the respirator and the user's face. This seal is critical in preventing the ingress of harmful particulates, pathogens, or hazardous substances. However, achieving and maintaining an effective fit is a persistent challenge due to variations in facial morphology, user movement, and environmental conditions.

Current fit testing methods fall into two categories: qualitative fit testing and quantitative fit testing. Qualitative methods require subjective user responses to taste or odor stimuli, making them unreliable and unsuitable for individuals with impaired senses or biases. Quantitative methods, which commonly utilize particle counters, provide objective data but are often prohibitively expensive, require trained personnel, and are impractical for widespread use during public health emergencies or in resource-limited settings. Furthermore, neither method provides real-time monitoring during respirator use, leaving users vulnerable to face seal issues caused by movement or extended wear during public health emergencies.

The increasing frequency of public health crises, such as the COVID-19 pandemic, has underscored the critical need for an accessible, cost-effective, and continuous fit assessment solution. Despite advancements in wearable technology, existing systems for respirator fit evaluation remain bulky, complex, and can be limited to use only under controlled conditions.

The invention described herein addresses these limitations by introducing a Wearable Respirator Fit Assessment System. This system integrates multimodal sensing technology with machine learning to provide real-time, continuous feedback on respirator fit. The compact, user-friendly device is designed to empower individuals—both trained professionals and the general public—to achieve and maintain optimal respiratory protection in a variety of environments.

The proposed system represents a significant advancement over existing technologies by:
1. Eliminating the need for consumables and expensive particle counters.
2. Offering on-device fit evaluation (such as through machine learning), reducing dependency on external equipment.
3. Supporting continuous monitoring during respirator use, enabling users to respond immediately to face seal compromise.
4. Ensuring compatibility with a broad range of users and respirator models through robust training datasets.

By addressing these critical challenges, this invention has the potential to revolutionize the field of respiratory protection and improve safety outcomes for a wide range of users.

### SUMMARY

Accordingly, embodiments of the present disclosure include an improved wearable respirator fit assessment system and method for performing respirator fit tests and continuous respirator fit monitoring. The system can detect signals from the various sensors in fluid communication with the respirator dead space, which is formed by the close apposition and sealing effect of the respirator to a user's face and subsumes the volume between a user's face and the boundary of the respirator. Upon a user breathing in and out, air is drawn across the respirator into and out of the dead space, changing the properties of the dead space volume, which can then be measured by sensors contained within the wearable respirator fit assessment system. This data can then be transformed and analyzed, allowing for respirator fit to be assessed and continuous respirator seal monitoring to be performed. Additionally, embodiments of the present disclosure may include communication between the system and a connected electronic device such as smartphone or tablet computer to enable user feedback, instruction on proper fit testing technique, and learning materials to be presented with testing as part of a digital learning system.

The above summary is not intended to describe each disclosed embodiment or every implementation. The Figures and the Detail Description, which follow, more particularly exemplify illustrative embodiments.

### BRIEF DESCRIPTION OF THE DRAWINGS

The disclosure may be more completely understood by the following detailed description in conjunction with the accompanying drawings, wherein like reference numerals designate like structural elements.

FIG. 1 is a perspective view of an exemplary embodiment of a wearable respirator fit assessment system while being worn by a user.

FIG. 2 is an external front view of an exemplary embodiment of a wearable respirator fit assessment system showing external features.

FIG. 3 is an external perspective view of an exemplary embodiment of a wearable respirator fit assessment system showing external features.

FIG. 4 is an external side view of an exemplary embodiment of a wearable respirator fit assessment system showing external features.

FIG. 5 is a cross-sectional view of an exemplary embodiment of a wearable respirator fit assessment system demonstrating one possible internal layout of components, including sensors and microcontroller.

FIG. 6 demonstrates communication between an embodiment of a wearable respirator fit assessment system and a connected/user device according to an embodiment of the disclosure.

FIG. 7 depicts an embodiment of a method for monitoring the fit of a respirator.

FIG. 8A-8B depicts a possible embodiment of a smartphone application interface and how a fit test may be conducted according to an embodiment of the disclosure.

FIG. 9A-9B depicts a method of installing a wearable respirator fit assessment system onto a respirator according to an embodiment of the disclosure, including the use of one embodiment of an installation tool to create a void in the respirator to facilitate mounting the system.

### DETAILED DESCRIPTION

Exemplary applications of wearable respirator fit assessment system assemblies according to the present disclosure are described in this section. Various examples are provided to add context to, and aid in the understanding of, the subject matter of this disclosure. It should be apparent to one having ordinary skill in the art that the present disclosure can be practiced without some or all of these specific details described herein. Further, various modifications and/or alterations can be made to the subject matter described herein and illustrated in the corresponding figures to achieve similar results or similar advantages, without departing from the spirit and scope of the disclosure.

Any scientific and technical terms used hereafter have meanings commonly used in the art unless otherwise specified. The definitions provided are to facilitate understanding of frequently used terms and are not meant to limit the scope of the present disclosure.

Unless otherwise described, any numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below or beneath other elements would then be above those other elements.

As used herein, when an element, component or layer for example is described as forming a “coincident interface” with, or being “on”, “connected to,” “coupled with”, “in contact with”, “separating” or “adjacent” another element, component or layer, it can be directly on, directly connected to, directly coupled with, in direct contact with, or intervening elements, components or layers may be on, connected, coupled or in contact with or separating the particular element, component or layer, for example. When an element, component or layer for example is referred to as being “directly on,” “directly connected to,” “directly coupled with,” or “directly in contact with” another element, there are no intervening elements, components or layers for example.

As used herein, “have”, “having”, “include”, “including”, “comprise”, “comprising” or the like are used in their open-ended sense, and generally mean “including, but not limited to.” It will be understood that the terms “consisting of” and “consisting essentially of” are subsumed in the term “comprising,” and the like.

The term “integral” refers to being made at the same time or being incapable of being separated without damaging one or more of the integral parts.

The term “respirator” refers to a device that is worn by a user to filter air before the air enters the person's respiratory system.

The term “facepiece” refers to the structure that fits at least over the nose and mouth of a person and that helps define an interior air space separated from an exterior air space.

Exemplary applications of wearable respirator fit assessment system assemblies according to the present disclosure are described in this section. Various examples are provided to add context to, and aid in the understanding of, the subject matter of this disclosure. It should be apparent to one having ordinary skill in the art that the present disclosure can be practiced without some or all of these specific details described herein. Further, various modifications and/or alterations can be made to the subject matter described herein and illustrated in the corresponding figures to achieve similar results or similar advantages, without departing from the spirit and scope of the disclosure.

The present disclosure describes various embodiments of a wearable respirator fit assessment system having an enclosure housing a sensor or multiple sensors, including a pressure sensor, as well as temperature sensor, and other sensors including gas sensors and humidity sensors. In various embodiments, the system can be mounted to a respirator either externally, internally, or partially externally and partially internally. The system can detect signals from the various sensors in fluid communication with the respirator dead space, which is formed by the close apposition and sealing effect of the respirator to a user's face and subsumes the volume between a user's face and the boundary of the respirator. Upon a user breathing in and out, air is drawn across the respirator into and out of the dead space, changing various physical properties of the dead space volume, which can then be measured by sensors contained within the wearable respirator fit assessment system. In some embodiments, this sensor data can then be passed into a data processing pipeline that can be running internally on the system or externally in a separate external processing unit. Respirator fit assessments and continuous fit checks can be performed via analysis of sensor signals. Various signals can then be given to the user, including visual, auditory, or haptic, to alert the user to the current respirator fit state. In some embodiments, sensor signal processing is performed on the system device, including running a data pipeline that utilizes machine learning inference on a pre-trained machine learning model on the system device itself. In certain embodiments, the system may connect with a paired electronic device, including a smartphone or tablet computer, and be operated remotely via a fit testing and/or continuous fit monitor application. Through the fit testing application, the user may be coached in how to properly perform a fit test, as well as utilize uni- or bi-directional signaling to allow the result of the fit test to be displayed to the user on the paired electronic device. In certain embodiments, the paired electronic device may provide notifications when respirator fit has been compromised. Additionally, the paired electronic device may provide real-time feedback to the user on their current respirator fit, instruction on proper fit testing technique, and learning materials to be presented with testing as part of a digital learning system.

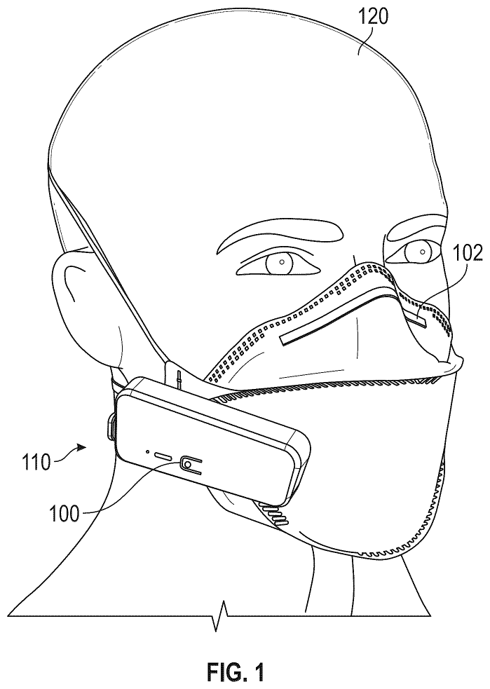

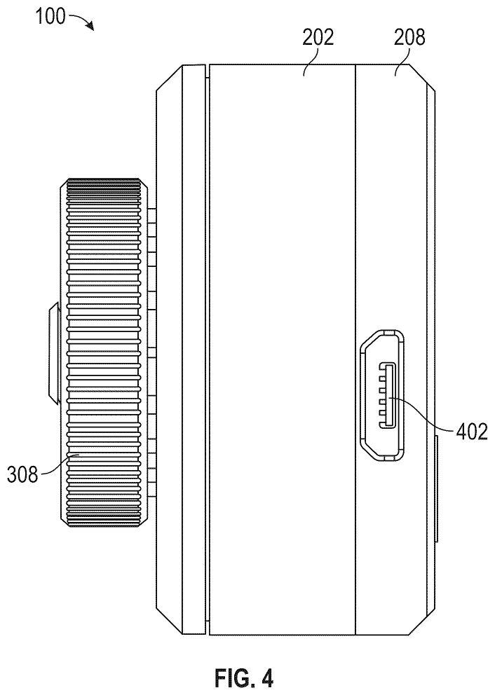

Referring to the drawings FIG. 1 to 9 illustrate a wearable respirator fit assessment system constructed according to an embodiment of the invention which is generally designated with the reference number 100.

FIG. 1 illustrates an exemplary embodiment of a wearable respirator fit assessment system 100 that may be attached to a respirator 102 worn by a user 120. In some embodiments, respirator 102 may seal against the face of a user 120 to cause air that is being breathed by the user to pass through the body of respirator 102, which can provide a filtering capability. The respirator 102 establishes a surface that partially defines the extent of an inner chamber and allows fluid communication between the outside environment and the user's respiratory tract, referred to as the dead space volume. In various embodiments, a wearable respirator fit assessment system 100 may interrogate this dead space volume to assess various physical changes occurring as a result of the user's breathing interacting with the partially-permeable respirator 102.

In an exemplary embodiment, the wearable respirator fit assessment system 100 may be mounted either to the outside or inside of a respirator 102 such that the visual indicator is visible in the user's peripheral vision, enabling the user to be alerted to detected changes in respirator fit. In some embodiments, the system 100 may include one or more light-emitting visual indicators that provide visual information in the form of various colors, flashing light patterns, or changes in light intensity to signal various information to the user while the device is being worn. The wearable respirator fit assessment system 100 may be attached to a respirator 102 either removably or permanently, using forms of affixation including threaded tube with threaded sealing flange, piercing mechanical connectors, hook and loop fasteners, liquid adhesive, pressure sensitive adhesive, magnets or other similar attachment methods.

Referring to FIG. 1, an external view is illustrated of an exemplary embodiment of wearable respirator fit assessment system 100. The wearable respirator fit assessment system 100 may be bounded by a device enclosure 202 enclosing various features, including a multifunction user interface button 204, a forward-facing visual indicator 206, and a peripheral visual indicator 208. In some embodiments, the multifunction user interface button may be used to turn the wearable respirator fit assessment system 100 on and off, cycle through various device modes, and silence nuisance alarms, among other functions. A forward-facing visual indicator 206 enables a user and others around them to clearly see the status indicator being displayed by the device when the device is not being worn or when a user is looking in a mirror when the device is being worn. A peripheral visual indicator 208 allows a user to clearly see the visual state of the device while it is being worn on a respirator by glancing down or by observing the peripheral indicator through the user's peripheral vision. In alternative embodiments, visual indicators may be omitted or configured to display in different orientations. Alternative embodiments may modify the user interface of the wearable respirator fit assessment system 100 by using capacitive touch surfaces or multiple tactile microswitches in lieu of the multifunction user interface button 204. Other embodiments may utilize a voice-controlled user interface using keyword and speech recognition to control various aspects of the device. Alternative embodiments may also provide feedback to the user using multiple methods, including visual, auditory such as with a buzzer or speaker, and haptic such as with a vibration motor.

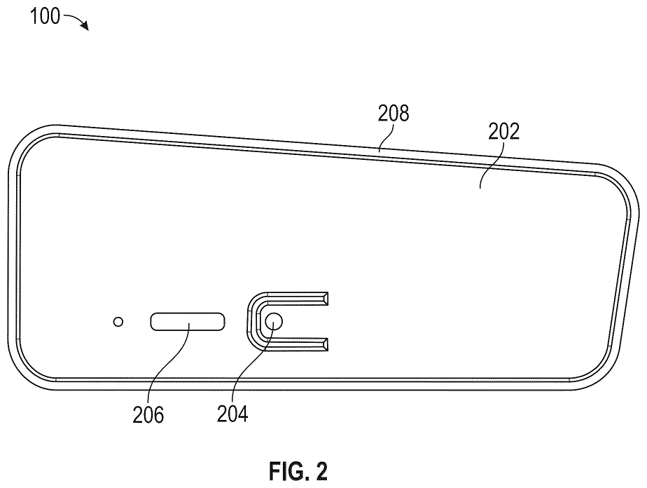

Referring to FIG. 2, an external perspective view is illustrated of an exemplary wearable respirator fit assessment system 100 demonstrating various aspects including one possible method of attachment using a threaded sample port 302 paired with a threaded sealing flange 308, which is inserted into a void created in the user respirator 102. In some embodiments, a sealing feature 304 may help provide an improved seal when a sealing flange 308 is tightened, trapping the respirator body between the sealing flange 308 and the sealing feature 304, enabling additional pressure to be exerted over the surface of the sealing feature 304. In addition, the sealing feature 304 may help prevent rotation of the device about an axis created by the sampling tube 302. Upon installation on a respirator, the sample tube orifice 306 is brought into fluid communication with the inside of the respirator, enabling sensors contained within a wearable respirator fit assessment system 100 to sample the respirator dead space and provide analysis and feedback to the user. The peripheral visual indicator in certain embodiments can provide visual indication of device state from various angles, which can improve the ability of a user to properly identify the visual indication in their peripheral vision.

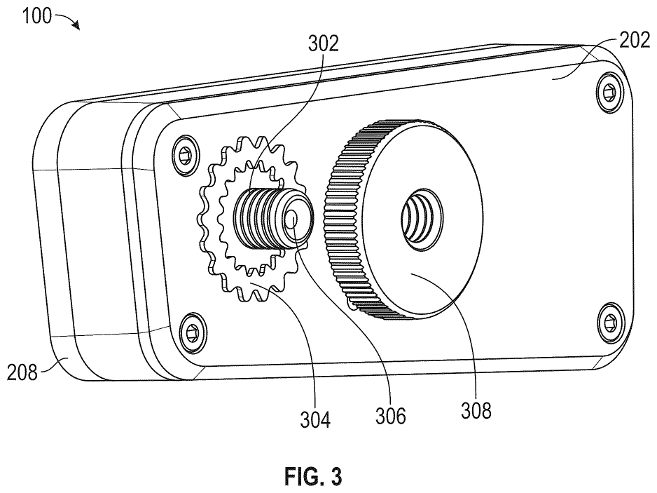

Referring to FIG. 3, an external side view is illustrated of an exemplary wearable respirator fit assessment system 100 which further demonstrates how the peripheral visual indicator 208 is visible on various surfaces of the device enclosure 202, improving a user's ability to discern the visual state of the device through their peripheral vision when the device is being worn. In some embodiments, a charge and data port 402 is present, enabling a user to recharge the device and connect the device directly to a computer for wired control of the device, to perform wired firmware updates, or to keep the device connected to power such as through a USB battery bank to enable charging and prolonged battery life, including while the device remains in use attached to a respirator 102.

In some embodiments, the wearable respirator fit assessment system 100 is attachable to a respirator or other type of respiratory mask. In other embodiments, the wearable respiratory fit assessment system 100 is integrally formed with a respirator or other type of respiratory mask where a sample port is in communication with an open space area or air volume of a body of the respirator or respiratory mask. In some embodiments, the respiratory fit assessment system 100 is formed on an external portion of the respirator or respiratory mask. In other embodiments, the respiratory fit assessment system is formed on an internal portion of the respirator or respiratory mask. Various tubings or channels may be configured within the respirator or respiratory mask that are communication with a volume of space of the respirator or respiratory mask when worn by a user. In some embodiments a sample channel extending from the housing. In some embodiments, the respiratory fit assessment system may be inserted inside of a respirator, or integrated into the respirator. For example, a gas mask may include a slot or receiver that that accepts the respiratory fit assessment system as a removeable or non-removeable sensing module.

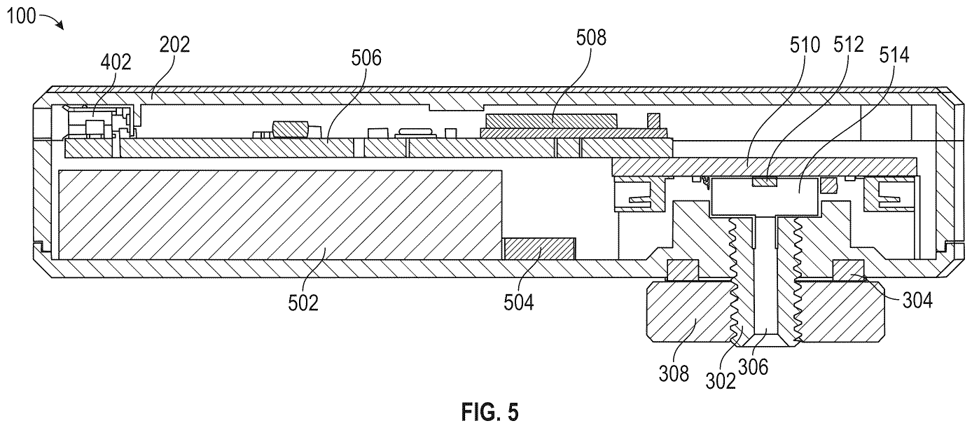

Referring to FIG. 5, a cross-sectional internal view is illustrated of an exemplary wearable respirator fit assessment system 100, demonstrating a possible device internal configuration and showing internal hardware. A device enclosure 202 may protect the internal hardware from physical damage including drops and impacts; liquid damage including rain, mist, humidity, and immersion; allow the device to be cleaned of dirt and pathogens to reduce the risk of fomite transmission; and to provide an enclosure that is easy for a user to physically grasp and install onto a respirator 102. In some embodiments, contained with the enclosure 202, may include a battery 502 which can be rechargeable (such as li-ion, lithium polymer, NiMH, among other chemistries) or primary/non-rechargeable (including lithium primary, alkaline, silver oxide, among other chemistries). Various embodiments may also include an auditory indicator 504 comprising a piezoelectric buzzer, speaker, electromechanical sounder, or other means of auditory signal transmission that can be used to alert a user. In certain embodiments, haptic feedback can also be provided through the use of linear or rotational vibration motors, providing various pulse sequences and at varying intensities to alert a user through haptic feedback conducted through sensory nerves. The wearable respirator fit assessment system 100 may utilize a printed circuit board (PCB) 506 for the embedded microcontroller (MCU) 508, as well as an additional PCB 510 for the sensor(s) 512. Some embodiments may use only one PCB which houses both MCU and sensor(s), or utilize one or more PCBs to house various components. Various embodiments may use one or more sensors 512 that may include sensors for pressure (absolute pressure or relative pressure sensors), temperature, humidity, gasses, particulates, acceleration (accelerometers), rotation (gyroscopes), magnetic fields (magnetometer), radiation, or sound intensity. In some embodiments, a sample chamber 514 is in fluid communication with sample tube 302 and sample tube orifice 306 to enable sensors 512 to access the respirator dead space volume.

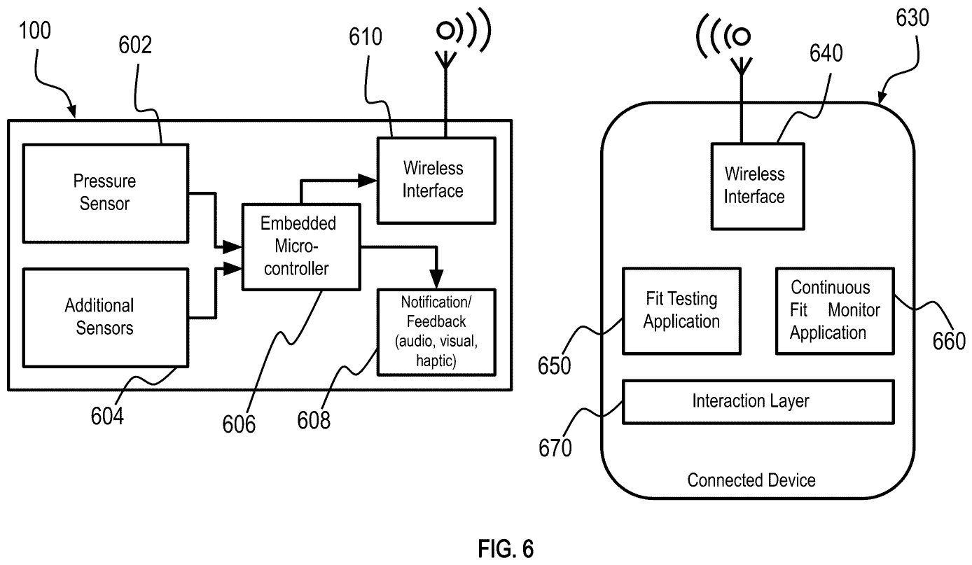

Referring to FIG. 6, communication between a wearable respirator fit assessment system 100 and a connected user device 630 is illustrated. In various embodiments, the connected user device may include a smartphone, tablet computer, personal computer, or smart watch, either owned by the user or supplied as part of the system. The pressure sensor 602 may utilize an absolute pressure sensor or relative pressure sensor in various embodiments. Additional sensors 604 may include temperature, humidity, gases, particulates, acceleration (accelerometers), rotation (gyroscopes), magnetic fields (magnetometer), radiation, sound intensity, or others. Pressure sensor 602 and additional sensors 604 may communicate with the embedded microcontroller 606, which in some embodiments may process signals from sensors with the output triggering either the wireless interface 610 and/or the notification/feedback mechanism 608, which may comprise audio, visual, and/or haptic feedback methods. In various embodiments, wireless interface 610 may include Bluetooth, Bluetooth Low Energy (BLE), or other radio protocols

In various embodiments, a connected device 630 may include a smartphone, tablet computer, personal computer, or smart watch that may contain a wireless interface 640, which could include Bluetooth, Bluetooth Low Energy (BLE), or other radio protocols. Wireless interface 640 may then enable communication with device wireless interface 610 in some embodiments to enable remote command and control of the wearable respirator fit assessment system 100. In some embodiments, wireless interfaces 610 may be configured as receive-only, transmit-only, or both receiver/transmitter (transceiver), as well as configured to send data unidirectionally or bidirectionally. In some embodiments, the notification/feedback system 608 may be used in combination with connected device speaker and microphone (not shown) to enable a non-radio datalink using information encoded in audio tones. In various embodiments, these tones may either be within the typical range of human hearing, or above the range of human hearing. In some embodiments utilizing audio-encoded data transmission, communication may be facilitated by not requiring prior Bluetooth pairing to be established. In some embodiments, wireless control may enable the user to perform a fit test using the fit testing application 650, which may include written and video instructions on how to properly perform a respirator fit test according to various standards, including various OSHA-validated fit testing methods, and to simultaneously allow the user to proceed through each fit test exercise and see the qualitative pass/fail or quantitative results of their fit test. In some embodiments, the passing fit test may then be communicated electronically in the form of an electronic fit test report. In various embodiments, a failing result may result in the user being provided with resources to understand why they may have failed their fit test, and the next steps to take in order to retest. In some embodiments, a continuous fit monitor application 660 may allow a user to receive updates on the status of their respirator fit in a continuous manner, while it is being worn, along with information on steps to take to improve fit if assessed fit is marginal or poor. In various embodiments, there may be an interaction layer 670 that allows a user to enter and receive information on the connected device 630.

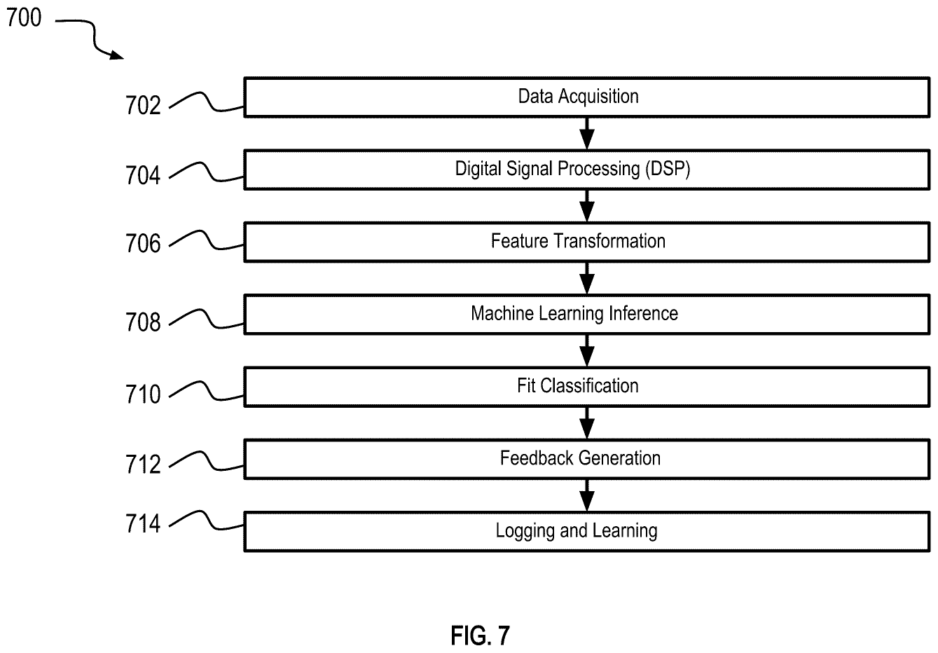

FIG. 7 illustrates an embodiment of a machine learning data processing pipeline. In various embodiments, data acquisition step 702 comprises sensor data continuously sampled from the respirator dead space utilizing sensor hardware to capture data at high temporal and special resolution along with transient changes indicative of fit issues. This acquired data may then be fed into a digital signal processing (DSP) step 704, where the data may be filtered for noise (e.g., removing environmental or electronic noise), signal normalization to standardize inputs across users, and feature extraction may be performed to isolate critical metrics. Processed data may then be fed into the feature transformation step 706, where in various embodiments, the filtered data is used to generate engineered features for model training and inference; including respiration rate patterns, transient anomalies indicative of respirator leaks, transient physiologic phenomena including coughing, sneezing, speaking, and sighing; resulting in a feature vector summarizing each data window. Transformed data may then undergo a machine learning inference step 708 where a pre-trained neural net is stored within the embedded microcontroller 606 and inferencing is conducted on-device to provide classification of data, including respirator fit states or anomalous conditions precluding valid assessment of respirator fit. After inferencing, data may then proceed to a fit classification step 710, where various categories or quantitative assessments of fit or anomaly may be generated. After fit classification, this data may then proceed to a feedback generation step 712 where a feedback mechanism or mechanisms are engaged, to provide visual (LED status indicator, green/yellow/red color-based indication), audio (sound alerts of varying pitch and/or intensity), haptic (vibrations of varying patterns and intensities), and/or via the wireless interface 610 to communicate fit information via the connected device 630. Next, data may proceed to a logging and learning step 714 so that data may be stored locally or transmitted remotely to improve machine learning performance over time, allowing continuous refinement of model performance for a broad range of user groups and edge cases.

In some embodiments, the machine learning model comprises one or more of: a neural network, a decision tree model, a gradient boosted model, a support vector machine, or another supervised learning classifier trained on labeled datasets. Moreover, the machine learning model may be a supervised, unsupervised, or semi-supervised model configured to classify respirator fit from sensor data.

In some embodiments, the system determines respirator fit using a sliding window method that evaluates sensor data in short, overlapping time segments. The sliding window method allows the device to provide continuous or near-continuous assessment of respirator fit and to detect changes that occur between formal fit test exercises or during routine use.

The wearable device collects real-time sensor data from the one or more sensors while the respirator is worn. The system divides the incoming data stream into a sequence of windows, each window representing a fixed period of time. Example window durations can range from 0.5 seconds to 5 seconds. The system can shift the start of each window forward by a step size shorter than the window duration to create overlap between adjacent windows. Example step sizes can range from 0.1 seconds to 1 second. These values are examples only and the system can employ any window size or step size suitable for the sensing modality and the processor constraints.

For each window, the system can process the sensor data in two general ways. In some embodiments, the system computes a set of features from the sensor data, including mean pressure, variance of pressure fluctuations, temperature gradients, respiration cycle amplitude, leakage signatures, airflow pulsatility, or other features that characterize signal changes associated with respirator fit. These computed features are then provided to a classification model.

In other embodiments, the system provides raw sensor values from the window directly to the classification model without feature extraction. The raw values can include pressure traces, temperature traces, airflow patterns, or other time series signals collected from the sensors. The classification model can be designed or trained to operate directly on raw data and can include a neural network, a temporal convolutional model, a recurrent model, a transformer based model, or any other model capable of handling sequential inputs.

The classification model outputs a window-level fit state. The system can update the current fit classification by aggregating the results of several recent windows. Aggregation examples include majority vote, weighted vote, confidence averaging, or temporal smoothing. This allows rapid detection of true changes in fit while reducing the impact of transient noise.

This sliding window method enables the device to monitor respirator fit continuously and to detect short-duration leaks that may not be captured by single exercise level evaluations. The method supports real-time alerts when the fit transitions from one state to another during normal breathing, movement, speech, or work activities. The method can be implemented on the wearable device, on a connected client device, or across both.

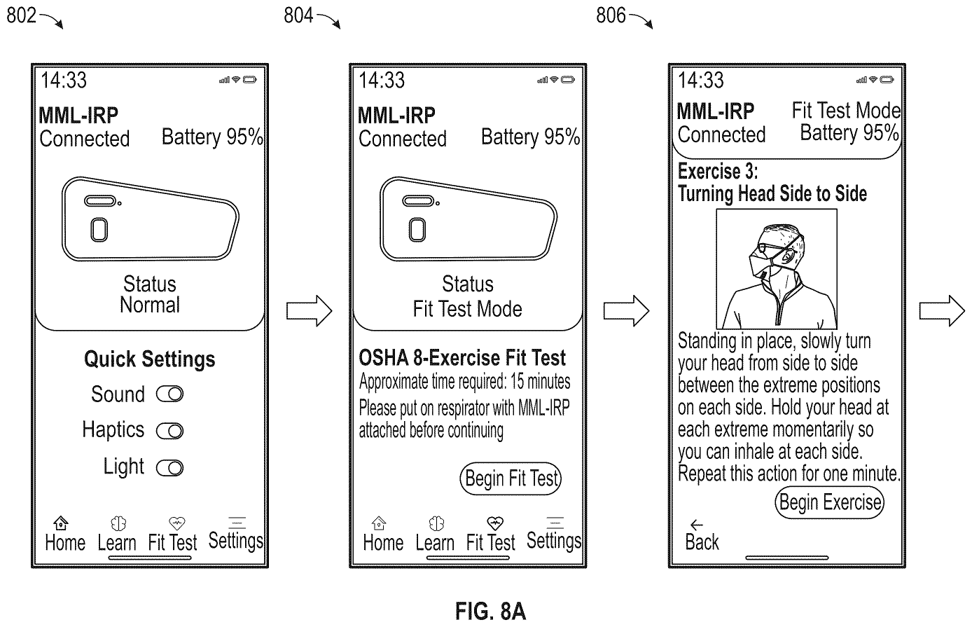

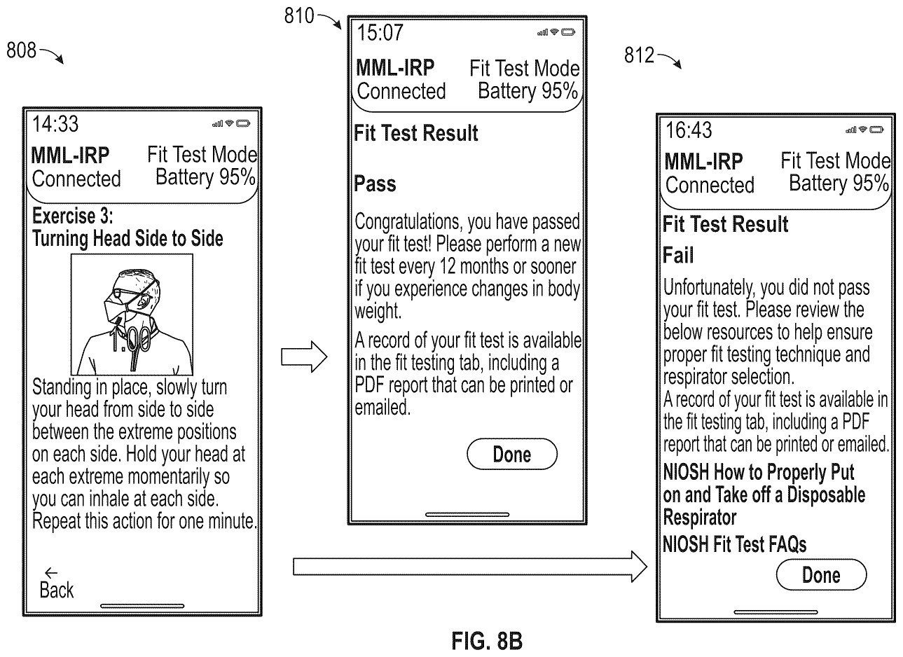

FIG. 8A-B illustrate exemplary embodiments of a fit testing application 650. After opening the fit testing application 650 on the connected device 630 using the interaction layer 670, the user may be presented with a screen such as in step 802. This screen may allow the user to configure wearable respirator fit assessment system 100 settings such as disabling audio, visual, and/or haptic feedback mechanisms, as well as initiating pairing to wirelessly connect the connected device 630 to a wearable respirator fit assessment system 100. In step 804, the user may select to run an OSHA-approved fit test using the connected wearable respirator fit assessment system 100. In step 806, the fit testing application 650 provides written and video guidance on how to properly perform each fit test exercise, along with a button to begin that exercise. Step 808 demonstrates an in-progress fit testing exercise, with the video and written guidance displayed on the connected device 630 screen, along with a countdown timer to help users properly perform each fit testing exercise. As a user proceeds through each fit testing exercise, the wearable respirator fit assessment system 100 may communicate with the connected device 630 after each exercise or at the end of all completed fit test exercises to update the connected device 630 with data on the pass/fail and/or quantitative fit assessment of each fit testing exercise. A fit test application 650 or a wearable respirator fit assessment system 100 may then process a composite score resulting in a final pass/fail fit assessment. After proceeding through all fit testing exercises, the user may then be presented with either a passing result in step 810 or a failing result in step 812, with guidance on remediating a failed fit test. After a passing result in step 810, a user may be prompted to electronically send a fit test record, if necessary.

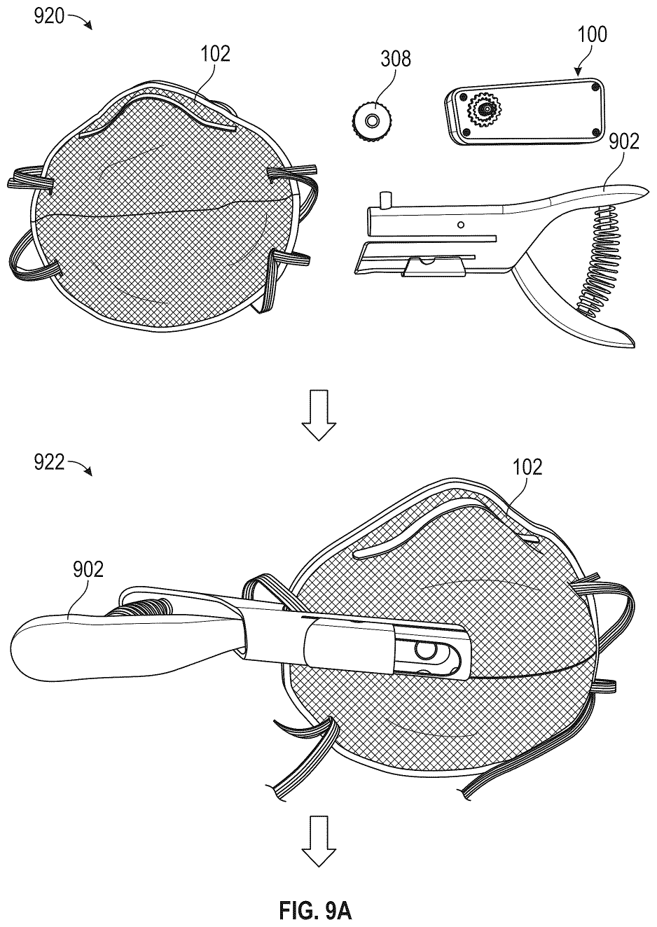

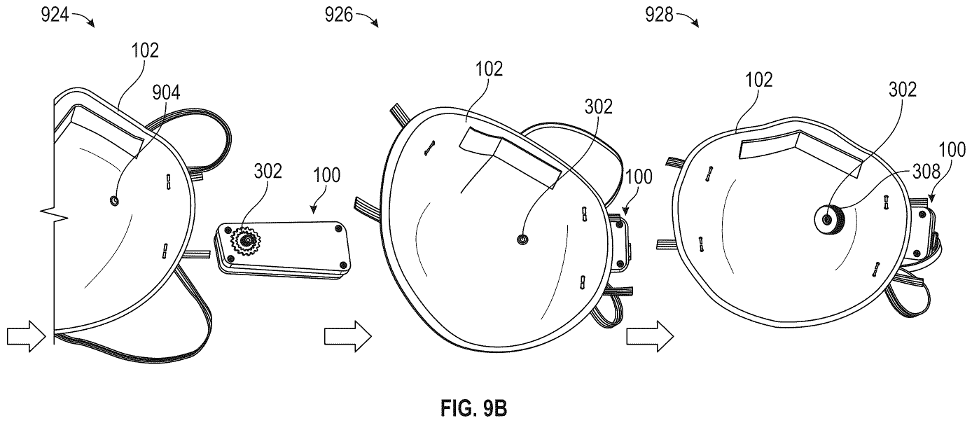

FIG. 9A-B illustrate an exemplary embodiment of an installation tool and method for installing a wearable respirator fit assessment system 100 on a respirator 102. In step 920, various items are gathered, including an installation tool 902, the wearable respirator fit assessment system 100, the sealing flange 308 and a respirator 102. In step 922, the installation tool 902 is positioned over the respirator 102 to create a void 904 in the specified location, and the installation tool is then actuated (not shown) which creates the void 904 in respirator 102. In step 924, the void 904 is inspected and shown to be patent, allowing the wearable respirator fit assessment system 100 to be installed. In step 926, the wearable respirator fit assessment system 100 is brought into close apposition with the respirator 102, with the user guiding sample port 302 through the void 904 in respirator 102. In step 928, the user places the sealing flange 308 over the sample port 302 and tightens the sealing flange 308 until it is finger-tight. In various embodiments, this may allow the wearable respirator fit assessment system 100 to be mounted to the respirator 102, with the sample port 302 in fluid communication with the respirator 102 internal dead space volume. The user may then follow manufacturer instructions and don the respirator.

### Reference Numerals

100 Wearable respirator fit assessment system
102 Example respirator the wearable respirator fit assessment system is mounted to
110 Wearable respirator fit assessment system example mounted to respirator being worn by a user
120 User
202 System enclosure
204 Multifunction interface button
206 Forward facing visual status indicator
208 Peripheral visual status indicator
302 Sampling port
304 Sealing feature
306 Sample port orifice
308 Sealing flange
402 Power and data port
502 Battery
504 Audible indicator
506 Microcontroller circuit board
508 Embedded microcontroller
510 Sensor circuit board
512 Sensors
514 Sampling chamber
602 Pressure sensor
604 Additional sensors
606 Embedded microcontroller (MCU)
608 Notification/Feedback providing audio, visual, haptic feedback
610 Wireless/Bluetooth interface
630 Connected/User device
640 Wireless/Bluetooth interface
650 Fit testing application
660 Continuous fit monitor application
670 Interaction layer/user interface
700 Data processing pipeline
702 Data acquisition step
704 Digital signal processing (DSP) step
706 Feature transformation step
708 Machine learning inference step
710 Fit classification step
712 Feedback generation step
714 Logging and learning step
802 Initial interaction layer view step
804 Fit testing application mode selection view step
806 User fit testing instructions with video demonstration view step
808 User fit testing countdown timer with video demonstration view step
810 User interface demonstrating passing fit test result step
812 User interface demonstrating failing fit test result step
902 Installation tool
904 Void in respirator created by installation tool
920 Materials assembled for device installation step
922 Introduction of the installation tool onto the respirator to create a void step
924 Wearable respirator fit assessment system in close apposition of prepared respirator step
926 Introducing wearable respirator fit assessment system sampling port through void in respirator created by installation tool step
928 Tightening sealing flange onto sampling port to affix system to respirator step

The included descriptions and figures depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the disclosure. Those skilled in the art will also appreciate that the features described above may be combined in various ways to form multiple embodiments. As a result, the invention is not limited to the specific embodiments described above, but only by the claims and their equivalents.

It will be appreciated that the present disclosure may include any one and up to all of the following examples.
Example 1: A system for fit testing and monitoring a respirator, comprising: a respirator; a wearable device configured to attach to the body of the respirator, the wearable device comprising: one or more sensors configured to detect variations in pressure, temperature, or other physical properties within the dead space of the respirator; a microcontroller configured to process data from the one or more sensors; a machine learning model, implemented on the microcontroller, configured to classify respirator fit; one or more feedback mechanisms configured to provide fit status notifications to the user via visual, auditory, and haptic signals; and a communication module configured to transmit fit data to an application on a connected device, wherein the connected device is configured to give instructions to the user, display real-time fit test results, provide guidance for improving respirator fit, and store historical fit data.
Example 2. The system of Example 1, wherein the wearable device is further configured to continuously monitor the respirator fit during use and to alert the user when the fit classification transitions to a marginal or poor fit state.
Example 3. The system of any one of Examples 1-2, wherein the machine learning model is a neural network trained on labeled datasets representing a broad range of users, respirator types, and user behaviors
Example 4. The system of any one of Examples 1-3, wherein the feedback mechanisms include: a multicolor LED indicator positioned in the wearable device; an audio indicator configured to emit tones corresponding to fit classifications; a vibration motor configured to deliver haptic notifications for silent feedback; and notifications delivered via a connected device, each configured to communicate fit status or alerts to the user.
Example 5. The system of any one of Examples 1-4, wherein the application on the user device is further configured to: guide the user through an OSHA-approved fit test protocol comprising multiple exercises, including breathing, head movement, and speech; display fit classification results for each exercise in real time; and provide educational resources on proper respirator use and maintenance.
Example 6. The system of any one of Examples 1-5, wherein the wearable device is powered by a rechargeable battery, and the housing is designed to be compact, lightweight, and resistant to environment conditions.
Example 7. A method for fit testing and monitoring a respirator, comprising: attaching a wearable device to the body of a respirator, wherein the wearable device includes one or more sensors, a microcontroller, and a machine learning model; donning the respirator by the user; performing a fit test by collecting sensor data during a sequence of fit test exercises, including breathing, head movements, and speech; processing the sensor data using digital signal processing and feature extraction techniques; classifying the fit of the respirator using the machine learning model; providing real-time feedback to the user through visual, auditory, and haptic notifications based on the fit classification; and transmitting fit test results to an application on a user device for display and guidance.
Example 8. The method of Example 7, further comprising continuously monitoring the respirator fit during use by: collecting real-time sensor data; updating the fit classification dynamically; and alerting the user to fit degradation when the classification transitions to a poor fit state.
Example 9. The method of any one of Examples 7-8, wherein the application on the user device provides step-by-step instructions for completing the fit test exercises and displays fit classification results in real time.
Example 10. The method of any one of Examples 7-9, further comprising: storing fit test data and historical fit classifications on the user device; and providing educational resources on respirator selection, use, and maintenance within the application.
Example 11. The method of any one of Examples 7-10, wherein the machine learning model is trained on labeled datasets representing a broad range of users and environmental conditions, enabling robust classification under varying user behaviors and respirator types.
Example 12. The method of any one of Examples 7-11, wherein the wearable device automatically enters a low-power sleep mode when the respirator is removed and resumes active monitoring upon detection of respiration.
13. The method of any one of Examples 7-12, wherein the wearable device includes an integral sampling port with a superfine mesh filter to prevent contamination of the sensor chamber.
14. The method of any one of Examples 7-13, wherein the wearable device detects transient leaks and provides immediate feedback to the user for corrective action.
Example 15. A system for fit testing and monitoring a respirator, comprising: a wearable device comprising: a housing; a respirator sample channel; one or more sensors in fluid communication with the respirator sample channel; and one or more processors, disposed in the housing, configured to process data received from the one or more sensors and determine a respirator fit.
Example 16. The system of Example 15, wherein the one or more sensors identify variations in pressure of air, air temperature, or other physical properties within a volume of dead space of the respirator when worn by a user.
Example 17. The system of any one of Examples 15-16, wherein the respirator fit is determined by a machine learning model trained to classify the respirator fit based on input sensor data from the one or more sensors.
Example 18. The system of any one of Examples 15-17, wherein the machine learning model is a neural network trained on labeled datasets representing multiple users, respirator types, and user behaviors.
Example 19. The system of any one of Examples 15-18, wherein the wearable device further comprises: a light emitting device, a speaker and/or a haptic device disposed in the housing, wherein the one or more processors generate a fit status notifications via visual, auditory, and/or haptic signals.
Example 20. The system of any one of Examples 15-19, wherein the wearable device further comprises: a multicolor LED indicator positioned in the housing of the wearable device; an audio indicator configured to emit tones corresponding to fit classifications; and a vibration motor configured to deliver haptic notifications for silent feedback.
Example 21. The system of any one of Examples 15-20, wherein the wearable device is further configured to continuously monitor the respirator fit during use of the respirator and to alert a user when the fit classification transitions to a marginal or poor fit state.
Example 22. The system of any one of Examples 15-21, wherein the wearable device further comprises: an application operable on a connected device; and a communication module configured to transmit respirator fit data to the application, wherein the application displays via a user interface, instructions to a user, displays real-time fit test results, provides guidance for improving respirator fit, and/or store historical fit data.
Example 23. The system of any one of Examples 15-22, wherein the application is further configured to: guide the user through an OSHA-approved fit test protocol comprising multiple exercises, including breathing, head movement, and speech; display fit classification results for each exercise in real time; and provide educational resources on proper respirator use and maintenance.
Example 24. The system of any one of Examples 15-23, wherein the respirator sample channel comprises a hollow interior body with a threaded exterior portion of the body that receives an attachment sealer, and wherein the respirator sample channel extends from the housing.
Example 25. A method for fit testing and monitoring a respirator, comprising: attaching a wearable device to a body of a respirator, wherein the wearable device comprises: a housing; a respirator sample channel extending from the housing; one or more sensors in fluid communication with the respirator sample channel; and one or more processors, disposed in the housing, configured to process data received from the one or more sensors and determine a respirator fit; performing a fit test by the wearable device by collecting sensor data by the one or more sensors during a sequence of fit test exercises; determining, by the one or more processors, a respirator fit based on the collected sensor data; and generating an indication by the respirator device of the determined respirator fit.
Example 26. The method of Example 25, wherein the wearable device further comprises:
wherein the one or more sensors identify variations in pressure of air, air temperature, or other physical properties within a volume of dead space of the respirator when worn by a user.
Example 27. The method of any one of Examples 24-26, wherein the respirator fit is determined by a machine learning model trained that classifies the respirator fit based on input sensor data obtained by the one or more sensors.
Example 28. The method of any one of Examples 24-27, wherein the machine learning model is a neural network trained on labeled datasets representing multiple users, respirator types, and user behaviors.
Example 29. The method of any one of Examples 24-28, further comprising: providing real-time feedback to the user through visual, auditory, and haptic notifications based on the fit classification; and
   transmitting fit test results to an application operable on a client device for display and guidance.
Example 30. The method of any one of Examples 24-29, wherein the application is configured to provide step-by-step instructions for completing a fit test exercises and display fit classification results in real time.
Example 31. The method of any one of Examples 24-30, further comprising: storing fit test data and historical fit classifications on the client device; and providing educational resources regarding respirator selection, use, and maintenance within the application.
Example 32. The method of any one of Examples 24-31, further comprising: continuously monitoring the respirator fit during use by: collecting real-time sensor data by the one or more sensors; periodically updating the respirator fit classification based on processing of the collected real-time sensor data via a trained machine learning model; and generating an alert or notification regarding the respirator fit degradation when a classification of the respirator fit transitions from a first fit state to a second fit state.
Example 33. The method of any one of Examples 24-32, further comprising; determining when the wearable device is removed from a user based on a detected change in air pressure; and automatically entering a low-power sleep mode after the respirator is removed; and resuming active monitoring upon detection of respiration.
Example 34. The method of any one of Examples 24-33, further comprising: detecting transient leaks in the respirator worn by a user by detecting a change in monitored air pressure; and generating an alert or notification for a user to correct the position or placement of the respirator about the user's face.
Example 35. The system of any one of Examples 15-23, wherein the one or more processors detect and analyze respiratory cycles from the one or more sensors and use characteristics of the respiratory cycles to assist in determining respirator fit.
Example 36. The system of any one of Examples 15-23, 35, wherein the one or more processors extract one or more temporal or frequency domain features from sensor data, the features including at least one of: peak-to-trough pressure amplitude, breathing frequency, rise time, decay time, or spectral energy, and use the extracted features as inputs for fit classification.
Example 37. The system of any one of Examples 15-23, 35-36, wherein the one or more processors classify leak events by type or severity, the classification including categories such as transient leak, sustained leak, speech-associated leak, and movement-associated leak, and wherein the one or more processors generate different notifications or guidance depending on the classified leak type.
Example 38. The system of any one of Examples 15-23, 35-36, wherein the wearable device performs a baseline calibration routine prior to a fit test, the routine comprising capturing a baseline respiratory signature, confirming sensor connectivity, and storing baseline parameters used to normalize subsequent sensor measurements.
Example 39. The system of any one of Examples 15-23, 35-38, wherein the wearable device comprises a sensor interface configured to sample a respirator microenvironment when the wearable device is mounted to or integrated with a respirator.
Example 40. The system of any one of Examples 15-23, 35-39,wherein the one or more processors implement power management including entering a reduced sampling low-power mode when respiration is not detected and automatically resuming active monitoring upon detection of respiration or user interaction.

Example 41. The method of any one of Examples 24-33, further comprising, during a fit test, segmenting collected sensor data into discrete exercise intervals, computing fit classification for each interval, and producing a composite fit score derived from interval classifications according to pre-defined aggregation rules.
Example 42. The method of any one of Examples 24-33, 41, further comprising: continuously evaluating derived feature statistics over a sliding time window to detect short duration leak events and distinguishing those events from normal respiration or environmental perturbations.
Example 29. The system of any one of Examples 15-23, 35-40, wherein the application provides an interactive guided fit test flow that conditions progression through exercises on successful completion of prior exercises, provides contextual corrective instructions for each failed exercise, and logs user actions and corrections for later review.

The terminology used herein is for the purpose of describing particular aspects only and is not intended to be limiting of the disclosure. As used herein, the singular forms “a”, “an”, and “the” are intended to comprise the plural forms as well, unless the context clearly indicates otherwise. It will be further understood that the terms “comprises”, “comprising”, or a combination thereof, when used in this specification, specify the presence of stated features, integers, steps, operations, elements, and/or components, but do not preclude the presence or addition of one or more other features, integers, steps, operations, elements, components, and/or groups thereof.

Although the previous disclosure has been described in detail by illustration and example for purposes of clarity and understanding, it should be recognized that the above disclosure can be embodied in numerous other specific variations and embodiments without departing from the spirit or essential characteristics of the disclosure. Certain changes and modifications may be practiced and it is understood that the disclosure is not to be limited by the foregoing details, but rather is to be defined by the scope of the following claims.

What is claimed is:

1. A system for fit testing and monitoring a respirator, comprising:
a wearable device comprising:
a housing;
a respirator sample channel;
one or more sensors in fluid communication with the respirator sample channel; and
one or more processors, disposed in the housing, configured to process data received from the one or more sensors and determine a respirator fit.

2. The system of claim 1, wherein the one or more sensors identify variations in pressure of air, air temperature, or other physical properties within a volume of dead space of the respirator when worn by a user.

3. The system of claim 1, wherein the respirator fit is determined by a machine learning model trained to classify the respirator fit based on input sensor data from the one or more sensors, wherein the machine learning model comprises a supervised, unsupervised, or semi-supervised model configured to classify respirator fit from sensor data.

4. The system of claim 3, wherein the machine learning model is trained on labeled datasets representing multiple users, respirator types, and user behaviors.

5. The system of claim 1, wherein the wearable device further comprises:
a light emitting device, a speaker and/or a haptic device disposed in the housing, wherein the one or more processors generate a fit status notifications via visual, auditory, and/or haptic signals.

6. The system of claim 1, wherein the wearable device further comprises:
a multicolor LED indicator positioned in the housing of the wearable device;
an audio indicator configured to emit tones corresponding to fit classifications; and
a vibration motor configured to deliver haptic notifications for silent feedback.

7. The system of claim 1, wherein the wearable device is further configured to continuously monitor the respirator fit during use of the respirator and to alert a user when the fit classification transitions to a marginal or poor fit state.

8. The system of claim 1, wherein the wearable device further comprises:
an application operable on a connected device; and
a communication module configured to transmit respirator fit data to the application, wherein the application displays via a user interface, instructions to a user, displays real-time fit test results, provides guidance for improving respirator fit, and/or stores historical fit data.

9. The system of claim 1, wherein the application is further configured to:
guide the user through a fit test protocol comprising multiple exercises;
display fit classification results for each exercise in real time; and
provide educational resources on proper respirator use and maintenance.

10. The system of claim 1, wherein the respirator sample channel comprises a hollow interior body with a threaded exterior portion of the body that receives an attachment sealer, and wherein the respirator sample channel extends from the housing.

11. A method for fit testing and monitoring a respirator, comprising:
attaching a wearable device to a body of a respirator, wherein the wearable device comprises:
a housing;
a respirator sample channel;
one or more sensors in fluid communication with the respirator sample channel; and
one or more processors, disposed in the housing, configured to process data received from the one or more sensors and determine a respirator fit;
performing a fit test by the wearable device by collecting sensor data from the one or more sensors during a sequence of fit test exercises;
determining, by the one or more processors, a respirator fit based on the collected sensor data; and
generating an indication by the respirator device of the determined respirator fit.

12. The method of claim 11, wherein the wearable device further comprises:
wherein the one or more sensors identify variations in pressure of air, air temperature, or other physical properties within a volume of dead space of the respirator when worn by a user.

13. The method of claim 11, wherein the respirator fit is determined by a trained machine learning model that classifies the respirator fit based on input sensor data obtained by the one or more sensors, wherein the machine learning model comprises a supervised, unsupervised, or semi-supervised model configured to classify respirator fit from sensor data.

14. The method of claim 13, wherein the machine learning model is trained on labeled datasets representing multiple users, respirator types, and user behaviors.

15. The method of claim 14, further comprising:
providing real-time feedback to the user through visual, auditory, and haptic notifications based on the fit classification; and
transmitting fit test results to an application operable on a client device for display and guidance.

16. The method of claim 15, wherein the application is configured to provide step-by-step instructions for completing fit test exercises and display fit classification results in real time.

17. The method of claim 15, further comprising:
storing fit test data and historical fit classifications on the client device; and
providing educational resources regarding respirator selection, use, and maintenance within the application.

18. The method of claim 10, further comprising:
continuously monitoring the respirator fit during use by:
collecting real-time sensor data by the one or more sensors;
periodically updating the respirator fit classification based on processing of the collected real-time sensor data via a trained machine learning model; and
generating an alert or notification regarding the respirator fit degradation when a classification of the respirator fit transitions from a first fit state to a second fit state.

19. The method of claim 7, further comprising;
determining when the wearable device is removed from a user based on a detected change in air pressure; and
automatically entering a low-power sleep mode after the respirator is removed; and
resuming active monitoring upon detection of respiration.

20. The method of claim 7, further comprising:
detecting transient leaks in the respirator worn by a user by detecting a change in monitored air pressure; and
generating an alert or notification for a user to correct the position or placement of the respirator about the user's face.

21. The system of claim 1, wherein the one or more processors detect and analyze respiratory cycles from the one or more sensors and use characteristics of the respiratory cycles to assist in determining respirator fit.

22. The system of claim 1, wherein the one or more processors extract one or more temporal or frequency domain features from sensor data, the features including at least one of: peak-to-trough pressure amplitude, breathing frequency, rise time, decay time, or spectral energy, and use the extracted features as inputs for fit classification.

23. The system of claim 1, wherein the one or more processors classify leak events by type or severity, the classification including categories such as transient leak, sustained leak, speech-associated leak, and movement-associated leak, and wherein the one or more processors generate different notifications or guidance depending on the classified leak type.

24. The system of claim 1, wherein the wearable device performs a baseline calibration routine prior to a fit test, the routine comprising capturing a baseline respiratory signature, confirming sensor connectivity, and storing baseline parameters used to normalize subsequent sensor measurements.

25. The system of claim 1, wherein the wearable device comprises a sensor interface configured to sample a respirator microenvironment when the wearable device is mounted to or integrated with a respirator.

26. The system of claim 1, wherein the one or more processors implement power management including entering a reduced sampling low-power mode when respiration is not detected and automatically resuming active monitoring upon detection of respiration or user interaction.

27. The method of claim 11, further comprising, during a fit test, segmenting collected sensor data into discrete exercise intervals, computing fit classification for each interval, and producing a composite fit score derived from interval classifications according to pre-defined aggregation rules.

28. The method of claim 11, further comprising: continuously evaluating derived feature statistics over a sliding time window to detect short duration leak events and distinguishing those events from normal respiration or environmental perturbations.

29. The system of claim 1, wherein the application provides an interactive guided fit test flow that conditions progression through exercises on successful completion of prior exercises, provides contextual corrective instructions for each failed exercise, and logs user actions and corrections for later review.
